(19) United States
(12) Patent Application Publication
(10) Pub. No.: US 2003/0004534 A1
(43) Pub. Date: Jan. 2, 2003

(54) Balloon transporter

(76) Inventors: Stephanie A. George, St. Louis Park, MN (US); Jeffrey A. Lechner-Riehle, Burnsville, MN (US); Kimberly A. Anderson, Eagan, MN (US); Patricia M. Derus, Rogers, MN (US); Steven T. Deininger, Savage, MN (US)

Correspondence Address: Oppenheimer Wolff & Donnelly LLP, 45 South Seventh Street, Suite 3300, Minneapolis, MI, 55402-1609, US

(21) Appl. No.: 10/162,144

(22) Filed: Jun. 3, 2002

Related U.S. Application Data
(60) Provisional application No. 60/295,307, filed on Jun. 1, 2001.

(51) Int. Cl. A61M 29/00

(52) U.S. Cl. 606/191

(57) ABSTRACT

A balloon transporter device, system and method for its use in deploying a detachable balloon at a target site in a patient is disclosed. The device includes an elongated tube having an inner lumen, an outer circumference and at least one opened end. The inner lumen is sized to receive a detachable balloon, and a proximal end adapted to retractably associate with a balloon delivery device. Alternatively, the device includes a housing configured as a truncated cone, the housing having an inner channel with decreasing diameters sized to accommodate a deflated balloon at its largest diameter and a catheter tip at its narrowest diameter. The balloon transporter protects the detachable balloon as well as aids in the application or loading of the detachable balloon onto a catheter tip. The balloon transporter further includes properties that increase the ease of use and stability of the device so as to reduce the effort required by the user during loading of a detachable balloon onto a delivery device.

### FIELD OF THE INVENTION

The present invention relates generally to a balloon transporter for protection and delivery of a detachable balloon in medical procedures. The present invention particularly relates to a balloon transporter to be used with a balloon delivery system when deploying detachable, inflatable balloons in medical procedures involving blood vessels, body cavities, treatment of vesicle ureteral reflux, fecal incontinence, gastrointestinal reflux, urinary incontinence, and the like.

### BACKGROUND OF THE INVENTION

Detachable balloons are used for a wide variety of medical procedures where occlusion of a vessel or duct, or compression at a target tissue site is desired. One specific example is in the treatment of urinary incontinence. In patients suffering from this condition, detachable balloons have been used to successfully treat the intrinsic sphincter deficiency. One or more detachable balloons are implanted into the tissue adjacent to the neck of the bladder of a patient. The inflated balloons aid in the ability of the sphincter muscles to occlude, which improves or resolves the condition. This method of treatment is preferred as it is minimally invasive, safe and less traumatic to the patient. As such, various systems have been developed for the delivery of balloon catheters to a desired target site. See, for example, U.S. Pat. Nos. 5,411,475; 4,802,479; and 4,686,962.

An example of such a system may be found in U.S. Pat. No. 5,830,228, which describes a device for delivering a detachable, inflatable balloon at a target site. The system includes a holder, a protective sheath configured for insertion through the holder and a catheter device for carrying a balloon. Use of the device requires loading of the balloon directly onto the catheter device by the user immediately prior to insertion of the balloon/catheter through the protective sheath. Once loaded, the balloon is exposed by retraction of the sheath portion of the delivery device.

It has been discovered that an obstacle or disadvantage to such systems is that the user himself/herself loads the balloon “naked” or bare onto the catheter. This requires handling of a small balloon as well as manipulation and positioning of the balloon valve onto the small catheter tip. Therefore, the risk of damage to the balloon during loading, or separation of the balloon from the catheter tip due to user misalignment is a limitation of these systems. Further, balloons manufactured for uses with such systems are often shipped with limited protection. This further increases the risk of delivering a damaged balloon to the user.

In view of the above, it is apparent that there is a need to provide a balloon transporter for protection and delivery of a balloon. Such a transporter can be used with a balloon delivery system with a more reliable and stable transport of inflatable, detachable balloons. There is also a need to provide a balloon transporter that is efficient, easy to use, reliable and functions as a holder. This would reduce the overall procedure time required and therefore reduce patient discomfort. Such a balloon transport device protects the balloon during shipping as well as reduces the preparation time required by the physician or clinician prior to a procedure. This reduces the overall medical procedure time, thus minimizing patient discomfort and trauma, as well as physician frustration.

### SUMMARY OF THE INVENTION

In view of the foregoing, it is an object of the present invention to provide a balloon transport device that protects and reduces the risk of damage to a balloon intended for delivery to a patient target site.

A further object of the invention is to provide a balloon transporter that is efficient, requires minimal effort by the user, is easy to use and is reliable.

A further aspect of the invention is to provide a balloon transporter that enhances the delivery or loading of a balloon onto a delivery device. Such an aspect can include a balloon transporter having a window for visual verification of correct loading of a balloon onto a delivery catheter.

In one aspect of the invention, a balloon transporter for use with a balloon delivery system for deploying a detachable, inflatable balloon to a target site in a patient is disclosed.

An additional aspect of the invention provides a method of using a balloon protector with a balloon delivery system for deploying a detachable, inflatable balloon at a target site in a patient in vivo. The system includes a balloon transporter configured for insertion through the lumen of the sheath assembly of a delivery device.

These and other objects not specifically enumerated herein are believed to be addressed by the present invention which contemplates a balloon transporter for protection and delivery of a balloon for inflation at a target site, the balloon transporter including a housing having a passageway sized so as to fit at least a portion of a detachable balloon.

### BRIEF DESCRIPTION OF THE DRAWINGS

FIG. 1 is a perspective view of a linear delivery system;

FIG. 2 is a detailed view of a balloon transporter in accordance with one embodiment of the invention;

FIG. 3 is an assembly view of a balloon transporter including a delivery device in accordance with one embodiment of the invention;

FIG. 4 is an enlarged view of the distal end region of a balloon transporter system;

FIG. 5 is a view of a balloon transporter during loading of a balloon in accordance with one embodiment of the invention;

FIG. 6 is an enlarged view of the distal end region of a balloon transporter during loading of a balloon in accordance with one embodiment of the invention;

FIG. 7 is a cross-sectional view of a transporter in accordance with one embodiment of the invention;

FIG. 8 is an enlarged side assembly view of a balloon transporter as inserted into a balloon delivery device;

FIG. 9 is an enlarged view of the proximal end region of a balloon transporter as assembled within the base portion of a sheath assembly in accordance with one embodiment of the invention;

FIG. 10 is a cross-sectional view of a balloon transporter engaged with a delivery catheter in accordance with an alternative embodiment of the invention;

FIG. 11 is an enlarged side assembly view of a balloon transporter inserted into a balloon delivery system in accordance with one aspect of the invention;

FIG. 12A is a cross-sectional enlarged view of a balloon transporter housing a balloon in accordance with one aspect of the invention;

FIG. 12B is a planar view of a base support engaged with a balloon transporter in accordance with one aspect of the invention;

FIG. 12C is a planar view of an alternative base support engaged with a balloon transporter in accordance with another aspect of the invention;

FIG. 13 is an enlarged view of a balloon housed within the balloon transporter in accordance with the present invention.

### DETAILED DESCRIPTION OF THE INVENTION

The present invention is a device for enhanced transport of a balloon usable with a balloon delivery system. The balloon transporter functions to protect the balloon prior to its use, including protecting the balloon during shipment of the device to a user. In addition, the transporter operates as a holder and an alignment tool during the step of loading the balloon onto a balloon delivery device. Therefore, the present invention can be used with a variety of balloon delivery devices. For example, a balloon transporter can be used to load a balloon onto the catheter element described in U.S. Pat. No. 5,830,228, the entire disclosure of which is hereby incorporated by reference. Alternatively, a balloon transporter may be used with available delivery devices such as used with the “UroVive” delivery device (available from American Medical Systems, Inc. Minnetonka, Minn., the current assignee of the present invention).

In particular, the transporter of the present invention may also be used with a linear delivery device, such as described in Applicant's co-pending U.S. patent application Ser. No. 09/547,952 filed on Apr. 12, 2000, entitled “Linear Delivery System For Deployment of a Detachable Balloon at a Target Site In Vivo,” the entire disclosure of which is incorporated by reference herein.

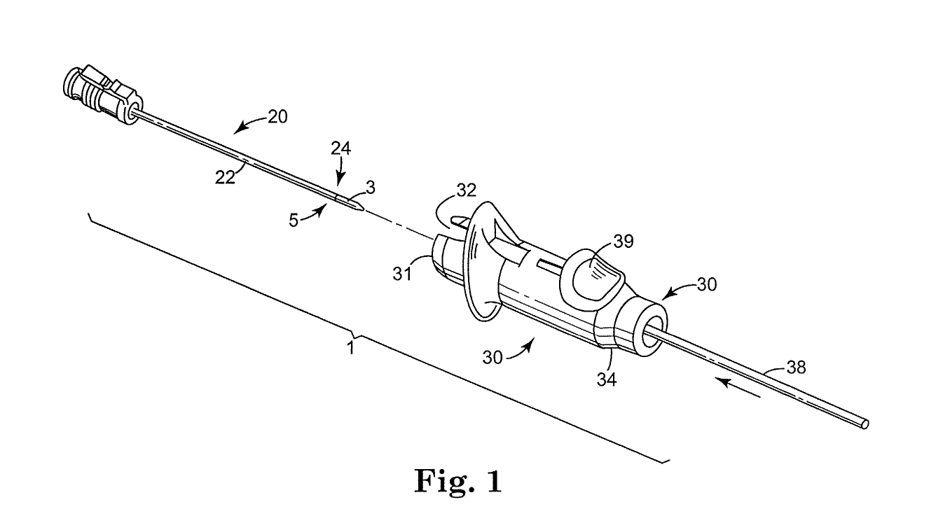

Referring to FIG. 1 and as described in the above-referenced co-pending Application, such a linear delivery system 1 includes a balloon delivery catheter 20 and a sheath assembly unit 30. When a linear delivery system 1 is used without a balloon transporter, the balloon delivery catheter 20 with a balloon 3 attached at its distal end 5, is inserted into the sheath assembly unit 30 through the proximal end lumen 32 of the sheath assembly unit 30 until the balloon is positioned within the shaft portion 38 of the unit 30. During such movement, the balloon 3 is exposed to the inside surfaces of the shaft portion 38. Subsequently, when the shaft portion 38 of the sheath assembly 30 is retracted (through rearward movement of the retracting tab 39), the detachable balloon 3 becomes exposed to the target site.

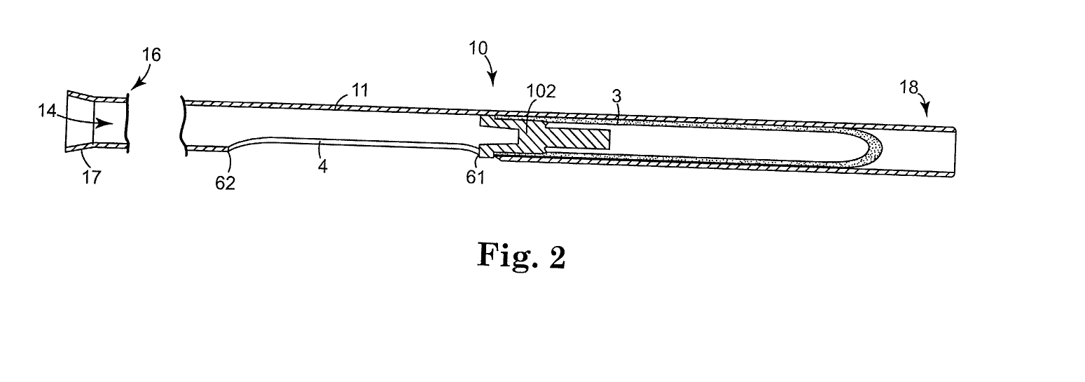

The present invention therefore provides enhanced transport of a balloon into the sheath assembly unit 30. Referring to FIG. 2, one embodiment of a balloon transporter 10 for use with a balloon delivery device or other similar device includes an elongated tube, sheath or sleeve 11 having an inner lumen 14 that is continuous between a proximal end 16 and a distal end 18 of the tube 11. The proximal end 16 of the elongated tube 11 is configured so as to mate with structure in the balloon delivery device (discussed below). The detachable balloon 3 is located or placed toward the distal end 18 of the inner lumen 14 of the elongated tube 11. Also, toward the distal end of the transporter 10 is an aperture 4 in the wall of the transporter 10. This aperture allows for visual observation of the balloon 3 as located in the transporter 10.

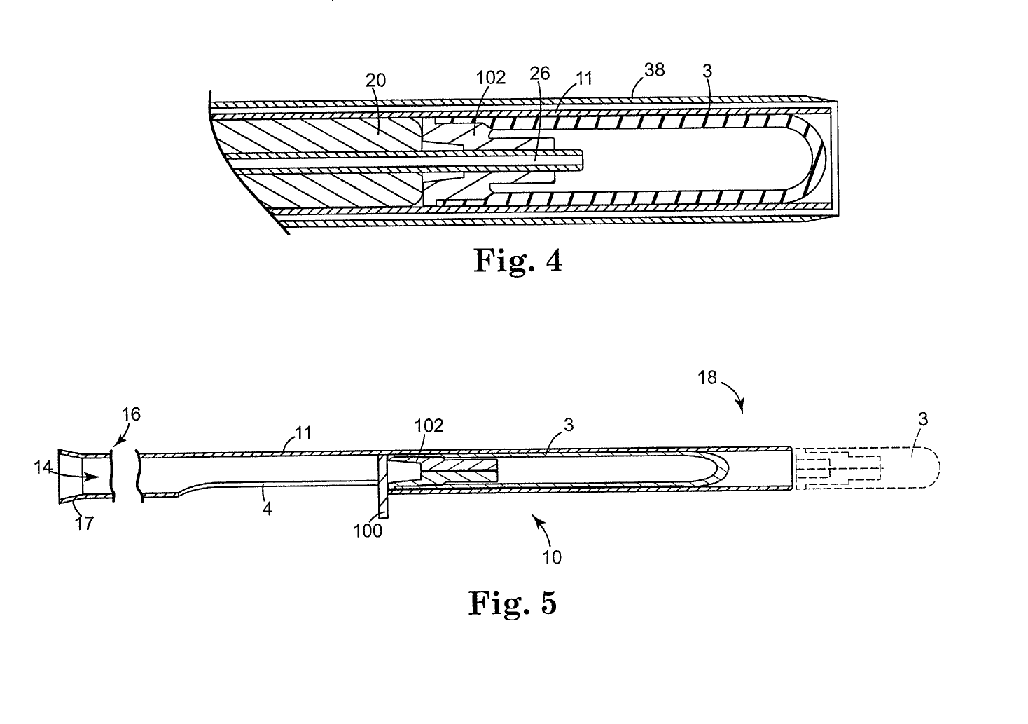

Referring to FIGS. 2 & 3, a balloon transporter 10 includes a tube or sleeve 11 which is elongated so as to fit over the catheter device 20 of a delivery system. Contained within the transporter 10 is the detachable balloon 3 for deployment at a target site in a patient. In use, the balloon transporter 10 is mounted on the shaft 22 of the catheter device 20 and slid along the catheter device 20 until the catheter tip 26 of catheter 20 engages and receives the detachable balloon 3. The resulting connection is shown in enlarged view in FIG. 4. Depending upon user preference, the balloon transporter 10 may be supplied preloaded, where the balloon is loaded into the transporter during manufacture, or can be manufactured “empty,” thus allowing the user to select a desired balloon for loading onto the transporter 10.

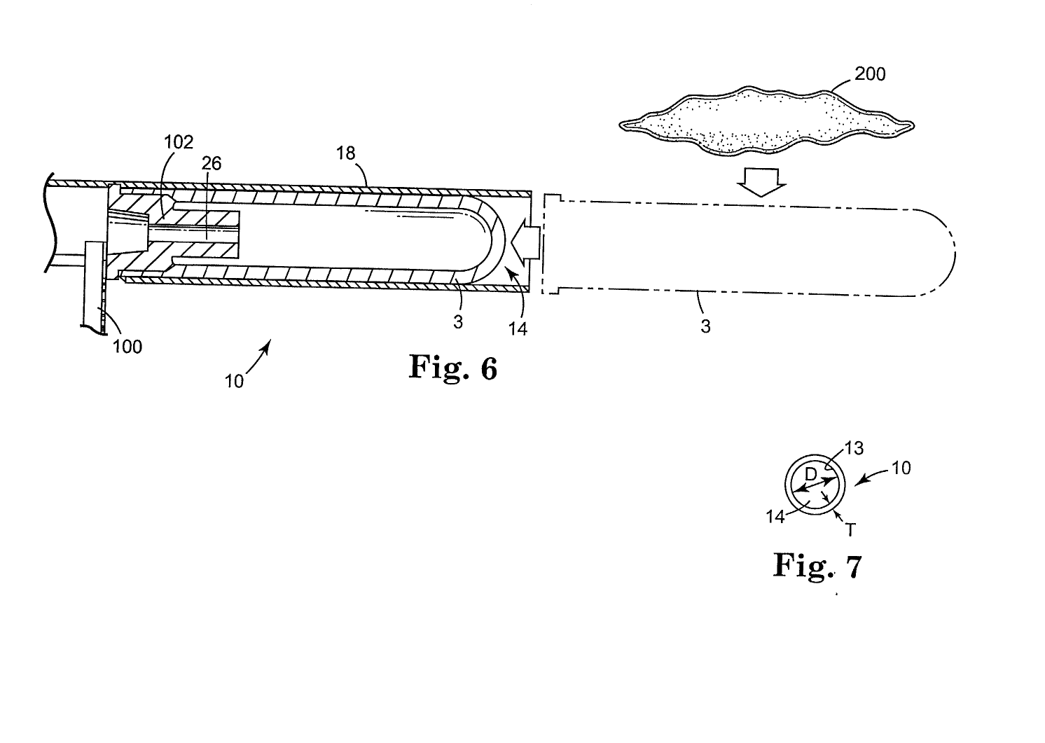

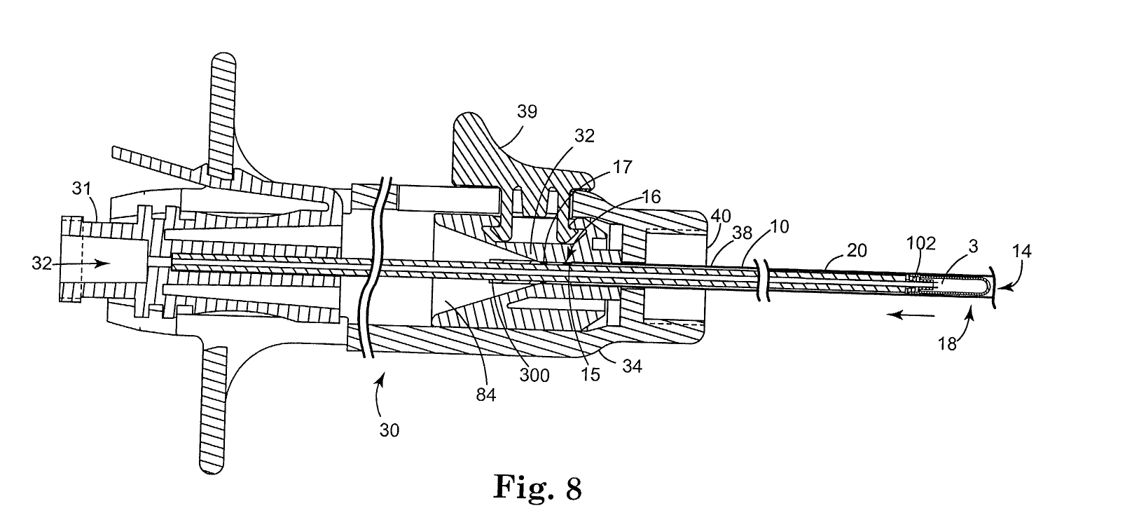

Referring to FIGS. 5 & 6, the loading of the balloon 3 into the transport 10 is now described. A stop member 100 is first inserted at the end of the aperture 4 nearest the distal end 18 of the transporter 10. The aperture 4 is then sealed with the stop member 100 in place. A vacuum is applied (not shown) at the proximal end 16 of the transporter 10. As shown in FIG. 6, a detachable balloon 3 (shown in phantom) lubricated with fluoro-silicone oil 200 is then introduced into the lumen 14 at the distal end 18 of the transport 10. The suction from the vacuum source (not shown) causes the detachable balloon 3 to move along the lumen 14 until it is prohibited from further movement by the stop member 100. Upon the balloon 3 arriving adjacent the stop member 100, the vacuum is discontinued and the seal and stop member 100 are removed from the aperture 4 (FIG. 5). In this state, the transporter is considered fully loaded and ready to transport the balloon 3 as described herein.

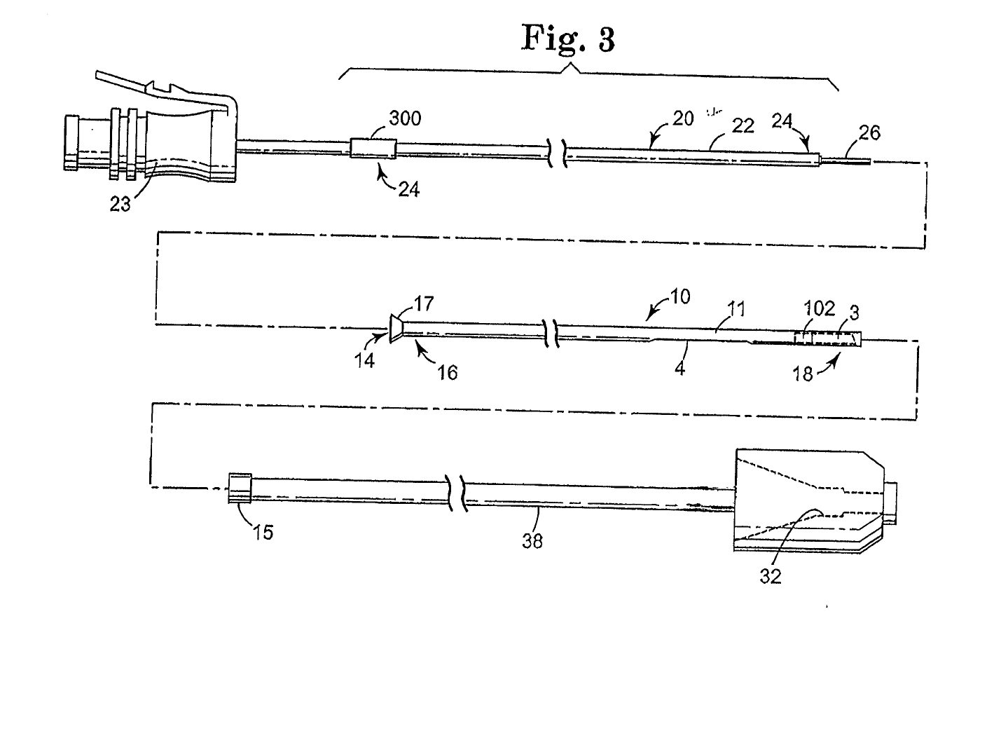

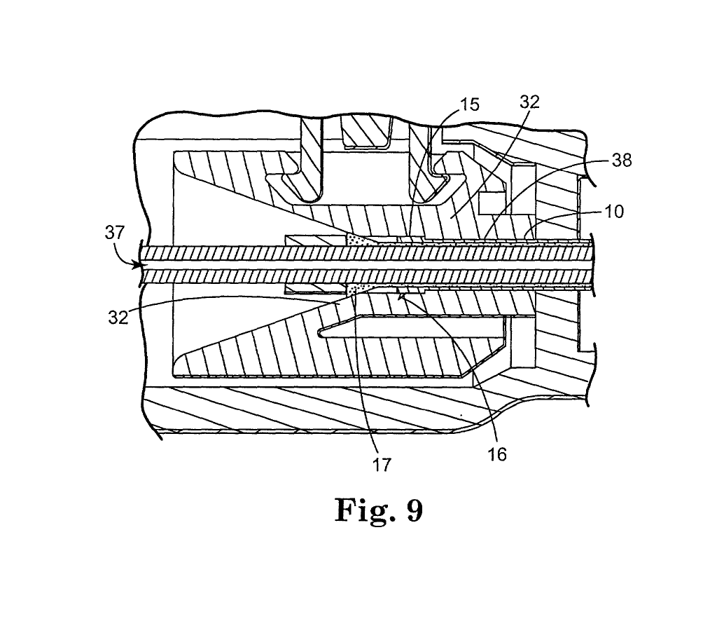

Returning to FIGS. 1 & 3, the tube 11 of a balloon transporter 10 (FIG. 3) should be of a length sufficient to adequately expose the balloon 3 when the proximal end 16 of the balloon transporter 10 is withdrawn into the base 34 (FIG. 1) of the sheath assembly 30 during deployment in vivo. The balloon transporter 10 should also be sized so that its inner lumen 14 passageway fits snuggly or closely over the shaft 22 of the catheter device 20. This close fit ensures that the catheter shaft 22 travels directly down the central axis or middle of the balloon valve and aids in aligning the balloon 3 onto the tip 26 of the catheter device 20 (FIG. 3). Referring to FIG. 7, in one embodiment, the diameter D of the transporter 10 is approximately 0.063 inches and the wall thickness T is approximately 0.0045 inches. In such an embodiment, the inner lumen diameter of the catheter shaft 22 (not shown) is approximately 0.060 inches.

In a preferred embodiment, when used with a delivery device as described in Applicant's co-pending U.S. Patent Application (supra), the balloon transporter 10 has an inner lumen diameter D that provides a slight compression fit for the detachable balloon. Referring to FIG. 7, the cross-sectional diameter D of the inner surface 13 of the balloon transporter 10 is smaller than the outer diameter of the detachable balloon 3. Therefore, in an embodiment where the detachable balloon has an outer diameter of about 0.07 inches (1.788 mm), the inner diameter D of the tube is about 0.063 inches (1.6 mm). This results in a balloon fit that is neither too loose nor too tight and serves to hold the balloon in place. An inner lumen 14 that provides a loose fit can cause the balloon contained within the tube to slide away from the tip of the catheter device during loading. A fit that is too tight can result in a balloon that does not easily move within the lumen and thus fails to properly deploy upon retraction of the balloon transporter 10 as described below.

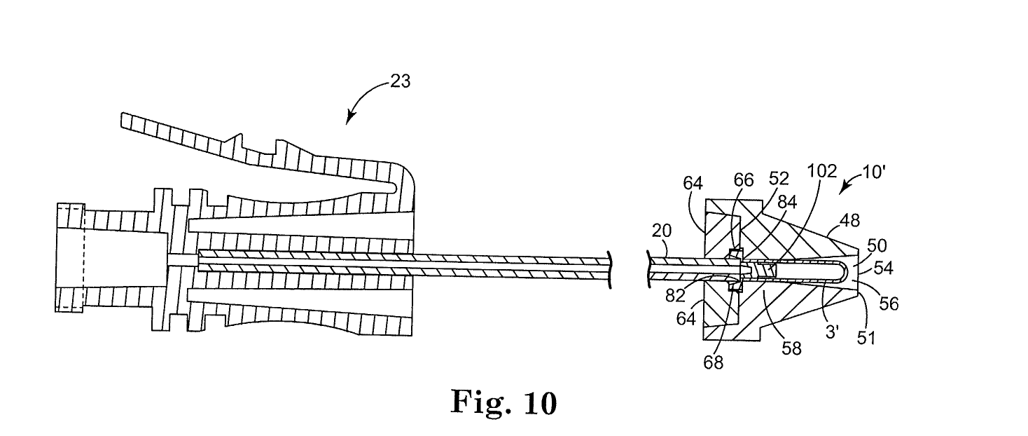

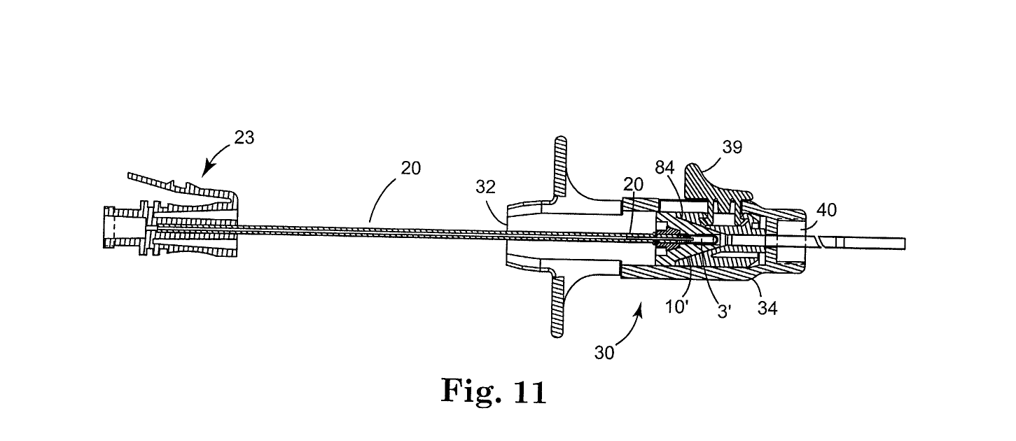

As shown in FIGS. 3 and 8, the sleeve 11 of a balloon transporter 10 has an inner lumen 14 or passageway that extends axially and is continuous between its proximal end 16 and distal end 18. Referring to FIGS. 8 & 9, the transporter 10 contains a flared portion 17 that extends radially outward from the longitudinal axis of the proximal end 16 of the transporter 10. In this embodiment, the flared portion 17 mates with the internal geometry of the sheath assembly unit shaft hub 31 of the delivery device when the balloon transporter 10 is inserted through the lumen 32 of the sheath assembly 30. The proximal end 15 of the shaft 38 is integrally coupled to the shaft hub 31 of the delivery device. Movement of the retraction tab 39 (FIG. 8) in the rearward direction causes the shaft hub 31, coupled to the proximal end 15 of the shaft 38 of the sheath assembly 30, to retract into base 34 of the sheath assembly 30. This also causes retraction of the balloon transporter 10 into the sheath assembly 30 by engaging the flared portion 17 of the transporter 10. The simultaneous pulling or withdrawing of the transporter 10 away from the target site exposes the detachable balloon 3 mounted on the distal tip 26 of the catheter 22 which was previously contained within the lumen of the balloon transporter 10.

Returning to FIG. 3, a balloon transporter 10 having a flared portion 17 is also advantageous in that the flared portion 17 aids the user in aligning the transporter 10 over the shaft 22 of the catheter 20 by operating as a centering surface edge. In other words, the shaft 22 of the catheter 20 is “funneled” into the lumen 14 of the transporter by the flared portion 17. This decreases the time and effort that would otherwise be required by the user when inserting the catheter 20 into the transporter 10 to load the balloon onto the catheter. Once placed over the catheter 20, the balloon transport 10 serves to guide the distal tip 26 of the catheter 20 into the base or valve region 102 of the detachable balloon 3 until the detachable balloon 3 is fully mounted onto the distal tip 26 of the catheter 20. The fully mounted configuration is shown in FIG. 4.

Referring to FIG. 2, as discussed previously, a preferred embodiment of the transporter 10 includes an aperture 4 for viewing the detachable balloon 3 or at least the base or valve region 102 of the detachable balloon 3 contained within the transporter 10. The location of the aperture 4 on the transporter 10 depends upon the shaft length of the balloon delivery structure. In a preferred embodiment, when used with a linear delivery device as disclosed in the previously identified co-pending patent application, the distal edge 61 of the aperture 4 is about 0.34 inch or 8.636 mm from the distal end 18 of the transporter. The proximal edge 62 of the aperture 4 is about 0.54 inch or 13.716 mm from the distal end 18 of the transporter. Therefore, the axial dimension of the aperture 4 is about 0.2 inch or 5.08 mm in length. These dimensions allow the user to see the base or valve region 102 of the balloon 3 if desired.

The ability to visualize the balloon provides an added level of assurance to the user when loading the balloon 3 onto the distal tip 26 of the catheter 20. In addition, where a transporter 10 is provided that is not preloaded with a detachable balloon 3, the aperture 4 may serve as an opening through which a detachable balloon 3 may be loaded into the transporter 10. In this latter case, the aperture should be of a size sufficient to allow insertion of a balloon 3 without causing damage to the balloon 3.

When provided as a verification mechanism, the aperture 4 should preferably be of a size that allows the user to visually see at least the base valve region 102 of the balloon 3 contained therein, yet still provides protection to the balloon 3. The aperture can be sized so as to have a width dimension 66 in the range of about 10% to 50%, or 25% 35%, or approximately one third, of the circumference of the tube 11. In a preferred embodiment, the aperture 4 will have a width of about 0.053 inch or 1.346 cm.

Referring to FIG. 6, the balloon transporter 10 can also include an inner surface that is lubricated so as to allow easy insertion of the balloon into the transporter 10 during manufacture as well as easy deployment of the balloon 3 at a target site. The lubricant 200 can be applied either to the balloon 3 or to the inner lumen 14 of the transporter 10. In a preferred embodiment, the lubricant material is a coating such as a fluoro-silicone oil, acetone, alcohol, or PEG solution with water. A fluoro-silicone oil is particularly advantageous when used with a silicone balloon insofar as the fluoro-silicone also prevents balloon adherence to the inner surface of the transporter tube 10. Without a fluoro-silicone lubricant, the silicone material of the detachable balloon 10, over time, can begin to adhere or stick to the inner surface of the transporter tube 10. Therefore, a fluoro-silicone oil helps to increase the shelf life of a balloon transporter/detachable balloon device.

As described above and shown in FIGS. 3 & 8, the transporter 10 with the catheter 20 mounted therein is placed within the catheter shaft of a sheath assembly 30, such as a linear delivery device (supra). As used with a linear delivery device, the balloon transporter 10 containing a detachable balloon 3 is first mounted over and onto the catheter device 20 until the distal tip 26 of the catheter 20 pierces the pre-pierced valve region 102 of the balloon 3. The transporter/balloon/catheter assembly is then inserted into the lumen 32 of the base 34 of the sheath assembly 30 (FIG. 8). As the catheter 20/transporter 10 is pushed down through the lumen 32 of the shaft portion 38 of the sheath assembly 30, the transporter 10 protects the balloon. The hub 23 of the catheter 20 (FIG. 3) then snaps into the sheath assembly base 34 (FIG. 8) when the correct position is reached. To expose the balloon 3, the tab 39 disposed on the sheath assembly 30 (which is connected to the shaft 38) is retracted. This also retracts the transporter 10 due to the flared portion 17 on the proximal end 16 of the transporter 11 engaging the shaft hub 32 of the shaft 38. This exposes the balloon 3 to the target site in vivo and the balloon is inflated. Once the balloon is deployed, the entire assembly (sheath, catheter and transporter) is removed from the patient.

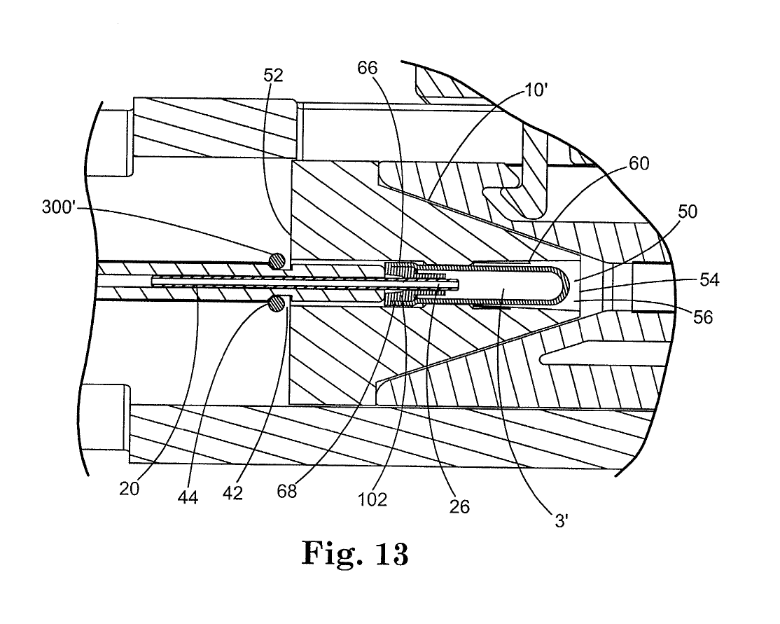

Referring to FIGS. 8 and 13, in another embodiment of the present invention, the balloon transport system includes a balloon transporter 10 and a stop element 300. The stop element 300 may be affixed to the shaft 22 of the catheter device 20 and is comprised of structure that operates to temporarily control and restrict the distance that the shaft 22 of the catheter 20 extends into transporter 10. It is preferred that such an element be temporarily controlling and/or restrictive so that after the stop has ensured correct alignment of the catheter shaft 22 (with the catheter tip engaging the balloon 3), the transport 10 may be urged or retracted beyond the stop element 300 as is necessary during use to expose the balloon 3 located on the distal tip 26 of the catheter 20. In this fashion, the stop element 300 controls relative movement of the transporter 10 and the catheter device 20 between a first position and a second position, the first position being that location where the catheter 20 is restricted from further insertion into the transporter 10 and the second position being that location beyond the stop element 300 upon deployment of the balloon 3.

In one embodiment, a stop element 300 can be constructed of a piece of tubing that is disposed on the outer surface of the catheter shaft 22. For example, a piece of Teflon® or tygon tubing can be heat-sealed to the outer surface of the catheter shaft. Upon sealing, a force of about 3 lb. (1.361 kg) on the transporter 10 is required to break the seal around the catheter shaft. This seal provides the temporary restriction preventing further movement of the catheter shaft 22 into the transporter 10. The sealing force of 3 lb. (1.361 kg) is generally equivalent to the force applied when inserting the transporter 10/catheter 20 into the base portion 32 of a sheath assembly 30. Therefore, the Teflon tubing functions as a releasable barrier which, when engaged by the transport 10, indicates to the user that the balloon 3 contained within the transporter 10 has been properly aligned onto the distal tip 26 of the catheter 20. Referring to FIG. 8, once inserted into the sheath assembly 30, the user pushes the catheter 20/transporter 10 assembly into position in the sheath assembly 30. This requires a force of about 3 lb (1.361 kg) which leads to the breakage of the Teflon seal. The Teflon seal now being broken, there is freedom for withdrawal movement of the transporter 10 over the catheter 20 to expose the balloon 3.

Referring to FIG. 13, in another embodiment of the present invention balloon delivery catheter 20 may include groove 42 that seats stop element 300′. Stop element 300′ generally comprises an O-ring. Groove 42 extends circumferentially around shaft 22 of balloon delivery catheter 20 Groove 42 may include a beveled or chamfered edge 44 or, alternatively, a straight cut edge (not shown). Groove 42 ensures that the balloon delivery device 10, 10′ of the present invention is correctly positioned on balloon delivery catheter 20 so that balloon 3 remains intact until it is delivered to the base 34 of sheath assembly unit 30. The groove 42 and stop element 300′ allow the distal tip 26 of catheter 20 to engage the valve region 102 of balloon 3, 3′ maintaining the balloon 3, 3′ in position in channel 50 or alternatively the inner lumen 14 of elongated tube 11. As the catheter/balloon transporter/balloon assembly is advanced into the shaft hub 31 of sheath assembly unit 30 and through the sheath lumen 32, the stop element 300′ slides out of groove 42 and is pushed along the shaft 22 of balloon delivery catheter 20 by the balloon transporter 10′ until balloon 3, 3′ is delivered to the distal end 40 of the shaft portion 38 of sheath assembly unit 30. Balloon 3, 3′ is now in position for deployment at the target site.

Examples of balloon types useful in the present invention are disclosed in U.S. Pat. Nos. 5,304,123, 5,411,475 and 4,311,146. Other balloons made of silicone or other elastomeric material may be used.

In a preferred embodiment of the present invention for use with a periurethral linear delivery system as described in Applicants' co-pending Application (supra), the shaft portion 38 of the sheath assembly 30 has a manufacturing specification of 2.750 inches (69.85 mm) in total length. The internal diameter of the shaft portion 38 is 0.077 inches (1.956 mm), or a size sufficient to allow passage of the transporter 10. Therefore, the balloon transporter 10 will preferably have a total length of about 2.760-inch (70.104 cm) with a flared portion 17 dimension of about 0.085 inch (2.159 mm) in diameter. The balloon transporter 10, 10′ in accordance with the present invention, may be made of a rigid material to protect the balloon during passage through the lumen of the sheath assembly. Alternatively, a flexible material that has sufficient rigidity so as to protect the balloon during passage through the lumen of the sheath assembly may be used. Representative examples of suitable materials include, nickel titanium, stainless steel, certain grades of nitinol, urethane, polycarbonate, acetyl, nylon, PTFE/teflon, polyimides, polyamides, polyethylene, polypropylenes and mixtures thereof. In a preferred embodiment, balloon transporter 10′ is made from any thermoplastic material that may be injection molded such as those thermoplastics enumerated previously.

In a preferred embodiment of a balloon transporter 10 for use with a transurethral system for delivery of a detachable balloon, the length of the transporter 10 will be adapted to correspond appropriately with the increased catheter 20 length of the balloon delivery device 30. In such uses, the balloon transporter 10 will preferably be fabricated of a more flexible materials such as polycarbonate, acetyl, nylon, urethane, silicone, teflon, polyimides, polyamides, polypropylene and nitinol in order to facilitate transport through the lumen of a cytoscope.

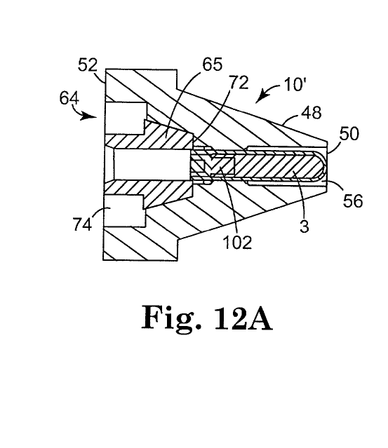

Referring to FIGS. 10 through 13, an alternative embodiment of the balloon transporter 10′ in accordance with the present invention is shown. Balloon transporter 10′ includes housing 48, channel 50, distal tip 51, and base 52. Housing 48 has the general configuration of a trapezoid or truncated cone. Channel 50 extends along the longitudinal axis of housing 48 from the distal tip 51 to base 52. Channel 50 includes two or more continuous sections of decreasing diameters. The largest diameter section 56 is located at the distal tip 51 and is dimensioned to accommodate the diameter of a deflated balloon. The middle section (not shown) of channel 50 is dimensioned to accommodate the diameter of the balloon valve region 102 and approximate the inside diameter of sheath 38. The narrowest section 58 of channel 48 at the base 52 is dimensioned to accommodate the diameter of distal tip 26 of catheter 20. The continuous decreasing diameters of channel 50 allow balloon 3′ to be correctly positioned without folding or buckling as may happen in a channel of uniform diameter. In addition, the junction of the narrowest section 58 and the middle section creates a ledge or platform 72 on which the valve 102 of balloon 3′ sits.

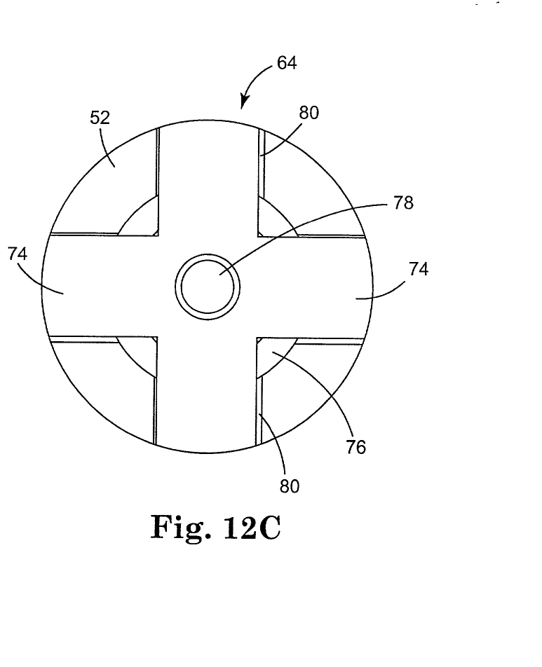

Referring to FIGS. 12B and 12C, housing 48 may also include base support 64. Base support 64 includes a central portion 76 including an aperture 78 located therewithin, and two or more flanges 74 integrally attached to central portion 76. The flanges 74 extend radially from base support 64 and are received in mating relationship with corresponding grooves 80 on base 52. Base support 64 snaps into mating relationship with grooves 80 of base 52. Alternatively, grooves 80 on base 52 may receive base support 64 in an interference fit relationship or base support 64 may be solvent bonded in grooves 80 or directly to base 52. Aperture 78 is aligned with pre-pierced valve 102 of balloon 3, 3′ and functions to additionally guide catheter tip 26 into valve 102. The two piece design of housing 48 and base support 64 facilitates placement of balloon 3, 3′ within channel 50 during manufacture and prior to use. Base support 64 also functions to restrain the balloon from dislodging from channel 50 during transport and prior to or while in use by the physician. Base support 64 may also include plug 65 which functions as an additional stop or restraint so that balloon 3, 3′ does not become dislodged after placement during manufacture or during use prior to being placed at the target site.

Housing 48 of balloon transporter 10′ includes a window or notch 60 that extends approximately 0.125 inches along the length of housing 48 and parallel to channel 50. Notch 60 is cut close to channel 50 leaving a thickness of approximately 0.010 to 0.012 inches. Notch 60 provides visualization of the balloon 3′ and distal tip 26 of catheter 20 and insures that during placement there are not any folds or twists in the balloon 3′. The ability to visualize the balloon 3, 3′ provides an added level of assurance to the user when loading the balloon 3, 3′ onto the distal tip 26 of catheter 20.

As noted previously, if balloon 3, 3′ is pre-loaded into the balloon transporter 10, 10′ during manufacture, the tip of the balloon 3, 3′ is dipped into a lubricant and passed through channel 50 of balloon transporter 10′. Valve 102 is also dipped into a lubricant. The use of a lubricant maintains balloon 3′ aligned in channel 50 without any deleterious breaks, twists or folds in balloon 3′. Alternatively, the inner surface of balloon transporter 10′ may be lubricated to allow easy insertion of the balloon 3, 3′ into transporter 10′. The lubricant material may comprise fluorosilicone oil, acetone, alcohol, or polyethylene glycol solution with water and mixtures thereof. A suitable lubricant for use in accordance with the present invention is fluorosilicone oil because it prevents balloon surface adherence to the inner surface of the transporter 10′ thus increasing the shelf life of a pre-loaded balloon 3, 3′.

One aspect of balloon 3′ may include balloon feet 66, 68. Balloon feet 66, 68 aid in the manufacture of balloon 3′ by providing a mechanism for the quick release of balloon 3′ from the manufacturing mold without damaging the fragile balloon3′ and be used for positioning balloon 3′ within transporter 10′. Additionally, balloon feet 66, 68 firmly seat balloon 3′ within transporter 10′. Upon deployment of balloon 3′ by catheter 20, balloon feet 66, 68 are sheared off by the cutting motion between edge 82 of catheter 20 and sharp edge 84 of ledge 72 in transporter 10′.

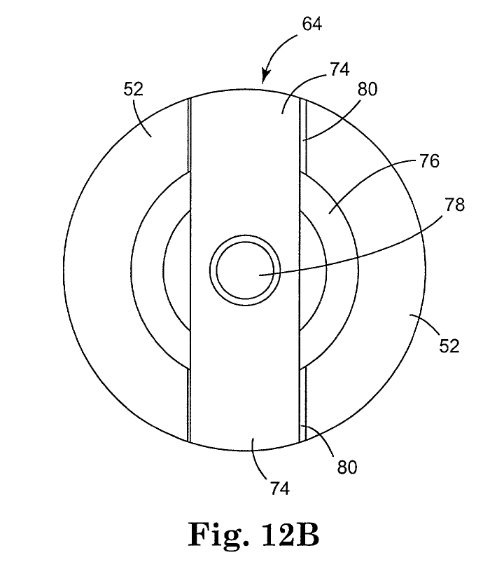

The in use operation of balloon transporter 10′ and the balloon delivery system of the present invention is substantially as described above for balloon transporter 10 and delivery system 1. As shown in FIG. 11, the transporter 10′ with the catheter 20 mounted therein is placed within the catheter shaft of a sheath assembly 30, such as a linear delivery device (supra). As used with a linear delivery device, the balloon transporter 10′ containing a detachable balloon 3, 3′ is first mounted over and onto the catheter device 20 until the distal tip 26 of the catheter 20 pierces the pre-pierced valve region 102 of the balloon 3, 3′. The transporter/balloon/catheter assembly is then inserted into the lumen 32 of the base 34 of the sheath assembly 30 (FIG. 11). The catheter 20 plus transporter 10′ including balloon 3, 3′ is pushed through lumen 32 of shaft portion 38 of sheath assembly 30. Transporter 10′ engages the conically-shaped portion 84 of lumen 32 and is prevented from moving forward as the catheter 20 and attached balloon 3, 3′ continue advancing toward distal end of shaft portion 38. The hub 23 of the catheter 20 (FIG. 11) then engagingly snaps into the sheath assembly base 34 (FIG. 8) at the same time the balloon 3, 3′ reaches the distal end 40 of shaft 38. To expose the balloon 3, 3′, the tab 39 disposed on the sheath assembly 30 (which is connected to the shaft 38) is retracted’. As balloon 3′ is advanced, transporter 10′ retracts along the length of catheter shaft 22. This exposes the balloon 3, 3′ at the target site in vivo and the balloon is inflated. Once the balloon is deployed, the entire assembly (sheath, catheter and transporter) is removed from the patient.

The present invention can be used for a variety of medical treatments where implantation of a detachable balloon 3, 3′ in a patient is desirable. For example, in the treatment of urinary incontinence, a detachable balloon 3, 3′ can be placed in the tissue immediately adjacent to the urethra. The inflated balloon 3, 3′ provides pressure external to the urethra, which causes partial closure of the urethra and reduces unwanted fluid leakage from the bladder through the urethra. Use of a balloon transporter 10 increases the efficiency and ease of use of such medical devices. Alternatively, the present invention can be used with other types of delivery devices or systems used to deliver a balloon to a patient.

The methods and systems of the present invention provide simple, accurate and stable transport of a balloon to a delivery system and a target site. The invention, as described herein with reference to preferred embodiments, provides a balloon transporter that protects and enhances the use of a detachable balloon in a medical procedure.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

All publications and patent applications in this specification are herein incorporated by reference to the same extent as if each individual publication or patent application was specifically and individually indicated by reference.

What is claimed is:

1. A transporter for a balloon comprising:
a housing having a proximal and distal end;
a lumen extending axially substantially along the length of said housing;
said lumen being sized to engage at least an external surface of said balloon and thereby retain said balloon in said lumen until said balloon is exposed for inflation at an intended target site.

2. The transporter as set forth in claim 1, wherein said housing is an elongated tube.

3. The transporter as set forth in claim 2, wherein a proximal end of said tube is shaped to have a diameter which is larger than an external diameter at substantially all other locations along said elongated tube.

4. The transporter as set forth in claim 2, wherein said proximal end of said tube is flared.

5. The transporter as set forth in claim 2, further comprising an aperture disposed in a distal region of said elongated tube.

6. The transporter as set forth in claim 5, wherein said aperture is sized to expose at least a base portion of said balloon.

7. The transporter as set forth in claim 1, further comprising a lubricant interposed between said external surface of said balloon and said lumen.

8. The transporter as set forth in claim 1, wherein said housing has a diameter sized to receive a catheter for engaging said balloon.

9. The transporter as set forth in claim 1, wherein said transporter comprises a rigid material.

10. The transporter as set forth in claim 1, wherein said transporter comprises a flexible material.

11. The transporter as set forth in claim 1, wherein said housing is configured in the shape of a truncated cone.

12. The transporter as set forth in claim 1, wherein said lumen comprises a uniform diameter.

13. The transporter as set forth in claim 1, wherein said lumen comprises varying diameters.

14. The transporter of claim 11, wherein said housing further includes a base support, said base support having two or more flanges matingly coupled with said proximal end of said housing.

15. A method of transporting a balloon intended for use in treating a medical condition comprising:
placing said balloon into a housing;
mounting said balloon onto an inflation catheter by inserting said inflation catheter into said housing until a distal end of said catheter engages said balloon;
inserting said housing and said inflation catheter into a balloon delivery sheath;
moving said housing relative to said inflation catheter and thereby exposing said balloon; and
detaching said balloon from said inflation catheter.

16. The method as set forth in claim 15, wherein said housing is an elongated tube.

17. The method as set forth in claim 16, wherein moving said elongated tube includes moving said delivery sheath.

18. The method as set forth in claim 16, wherein said balloon is inflated through said inflation catheter prior to detaching said balloon.

19. The method as set forth in claim 16, wherein placement of said balloon into said elongated tube includes holding said balloon in said elongated tube by frictional engagement between said tube and said balloon.

20. The method as set forth in claim 16, further comprising viewing said balloon in said elongated tube prior to inserting said elongated tube and said inflation catheter into said balloon delivery sheath.

21. The method as set forth in claim 15, wherein said inflation catheter is insertable into said housing a predetermined distance.

22. The method as set forth in claim 15, wherein said predetermined distance is controlled by a restrictive structure located on said inflation catheter.

23. The method as set forth in claim 15, wherein insertion of said inflation catheter and housing into said delivery sheath requires a force to overcome any resistive force caused by said restrictive structure.

24. The method as set forth in claim 21, wherein said force results in breakage of said restrictive structure.

25. A balloon delivery system for treatment of a medical condition comprising:
a balloon delivery assembly having a base and sheath, said sheath being movable relative to a balloon transporter containing a detachable balloon;
an inflation catheter insertable into said balloon transporter for attachment of said inflation catheter to said balloon;
said sheath having a diameter sized to receive said balloon transporter;
said inflation catheter being fixedly mountable to said balloon delivery assembly; and,
said sheath having a length so as to expose said inflation catheter upon movement of said sheath relative to said base.

26. The balloon delivery system as set forth in claim 23, wherein said balloon transporter is shaped to engageably mate with one end of said sheath such that movement of said sheath relative to said base in at least one direction causes movement of said balloon transporter.

27. The balloon delivery system as set forth in claim 24, wherein said balloon transporter comprises an elongated tube having a flared shape at one end to engageably mate with said one end of said sheath.

28. The balloon delivery system as set forth in claim 25, wherein said balloon transporter includes an aperture at a location to at least partially reveal said balloon.

29. The balloon delivery system as set forth in claim 23, wherein said balloon transporter comprises a housing configured in the shape of a truncated cone.
